(19) United States
(12) Patent Application Publication
(10) Pub. No.: US 2004/0161362 A1
(43) Pub. Date: Aug. 19, 2004

(54) Audible range acoustic cleaning process for implants

(76) Inventor: David L. Bogert, Alachua, FL (US)

Correspondence Address: DAVID L BOGERT, 14218 NW 188TH STREET, ALACHUA, FL, 32615, US

(21) Appl. No.: 10/679,470

(22) Filed: Oct. 7, 2003

Related U.S. Application Data
(60) Provisional application No. 60/420,025, filed on Dec. 27, 2002.

(51) Int. Cl. A61L 2/00; C23F 11/00

(52) U.S. Cl. 422/33; 422/1

(57) ABSTRACT

Human cadaver tissues have been used for many years to replace tissues in the body. These replacements not only include such well known organ transplants such as the heart and kidneys, they also involve soft tissue grafts such as tendons and arteries, and hard tissue grafts such as bone. It is desirable for these tissue grafts to be cleaned and sterilized. The basic technology to be utilized for this audible acoustic tissue cleaning process is the use of sound in a range audible to the human ear (20 Hz to 20,000 Hz) to provide the cleansing force necessary to clean and sterilize tissue grafts to be implanted into the human body. Another use of this technology is to clean and treat contaminated bone fragments from compound fractures. Another aspect of this audible acoustic tissue cleaning process is the use of relatively pure oxygen (50 to 100%) under pressure to treat the tissue grafts at 2 to 10 degrees Celsius.

### BACKGROUND

1. Field of Invention

This invention relates to the field of cleaning and permeating of tissue transplants with audible acoustic energy (or sound). Tissue transplants are medical grafts which utilize tissues from other humans, including cadavers (allografts), tissues from animals (xenografts), tissues from a person's own body (autografts) or contaminated bone fragments from compound fractures (also considered autografts).

2. Prior Art

LifeNet Research Foundation has five patents in this field. One patent is for cleaning large whole bones by applying suction to the interior of the bone and pulling a germicidal solution thought the bone walls (U.S. Pat. No. 5,556,379). The second LifeNet Research Foundation patent (U.S. Pat. No. 5,797,871) adds an ultrasonic cleaning bath to the process claimed in the first patent. This second patent on ultrasonics also claims to be useful to clean small tissues and bones in the absence of a vacuum. The recommended frequency of the ultrasonic sound is 40,000 to 47,000, a frequency well above the 20,000 Hz maximum frequency that a typical human ear can hear.

LifeNet Research Foundation has continued this line of patents with U.S. Pat. No. 5,820,581, U.S. Pat. No. 5,976,104 and U.S. Pat. No. 6,024,735, all dealing with the same basic processes. LifeNet Research Foundation's U.S. Pat. No. 5,556,379 (large scale pressure gradients) and LifeNet Research Foundation's U.S. Pat. No. 5,797,871 (ultrasonics), taken together, have large scale pressure gradients and ultrasonics working in tandem. These processes in tandem may have some degree of effectiveness in removing bone marrow from large bones and in removing some endogenous materials. The large scale pressure gradient cannot be applied to soft issues or machined bone components. Even in tandem, they apparently don't clean certain dense pockets of cortical very well. And either alone or in tandem, they apparently are not a reliable method of sterilization, at least not to the point of satisfying the FDA.

Osteotech has patents similar to the LifeNet Research Foundation's patent that use a vacuum applied to the inside of the bone to pull a solution through the bone (U.S. Pat. No. 5,513,662, U.S. Pat. No. 5,846,484 and U.S. Pat. No. 2002/00444888 A1). These do not mention ultrasonics. They would apparently have the same problems as LifeNet Research Foundation's main patent (U.S. Pat. No. 5,556,379).

Cryolife has a process (U.S. Pat. No. 5,333,626 and U.S. patent US2002/0128724 A1) which subjects the tissue to “high pressure washing conditions” with agitation or streams of water (no ultrasonics mentioned) subjecting the bone to 100 to 3000 psi. (preferably 500 to 1500 psi.). It apparently takes 1 to 2 days to complete, which would indicate high processing costs. It apparently would have the same problems as LifeNet Research Foundation's main patent (U.S. Pat. No. 5,556,379).

Regeneration Technologies Inc. has developed a patented process to sterilize allograft tissues (U.S. Pat. No. 6,482,584 B1 and U.S. Patent No. US 2002/0119437 A1). This process involves alternating pressure and vacuum being applied to the tissue with various cleaning and sterilizing solutions. Concurrent with the alternating pressure and vacuum, ultrasonic energy is applied. This process is apparently very capital intensive and expensive; as the alternating vacuum and pressure require some very heavy duty equipment (Regeneration Technologies Inc. press release of Jun. 12, 2002 mentions “several million dollars” for the equipment). And capital intensive operations are typically very expensive to run and typically result in high product manufacturing costs. And, as a rule, the more complex the equipment, the more difficult it is to maintain and properly control to FDA standards.

### OBJECTS AND ADVANTAGES

It is the objective of the audible acoustic range cleaning process for implants to afford tissue processors and hospitals the following advantages:

1. removes or kills viruses, fungi, bacteria and other potential disease organisms, thus being capable of sterilizing tissue to the current FDA definition of “sterility”

2. works for soft tissue and bone, whole bones or machined components

3. does not severely impact the strength of the implants

4. does not affect the ability of the donor tissue to act as a scaffold and encourages the growth of the host's tissue into the graft (osteoinductivity and osteoconductivity)

5. removes endogenous materials, including insoluble materials such cellular debris, from the transplants to prevent inflammatory and immune reactions

6, does not require millions of dollars of capital equipment, rather it only requires a small amount of equipment that can easily be installed and utilized in even a small tissue processing facility.

7, does not require large amounts of expensive sterile water and chemicals per treatment

8, has a treatment chamber small enough to be portable, allowing loading, unloading and chamber sterilization to take place other than in the treatment room or enclosure.

Technology Discussion

Human donor tissues (including cadaver tissues) have been used for many years to replace tissues in a living human body. These transplants not only include such well known organ transplants such as the heart and kidneys, they also involve non-organ transplants: soft tissues such as tendons, ligaments and arteries, and hard tissues such as cortical and cancellous bone. There were some 650,000 bone transplants in the year 2000 in the USA. Recently there have been at least 26 instances of infection (“sepsis”) and one death associated with these non-organ transplants, apparently due to bacterial contamination of the tissue transplants. Quoting United States Food and Drug Administration's Dr. Ruth Solomon (Blood Products Advisory Committee 72^{nd} Meeting, Mar. 15, 2002):

“there has been a recent report of sepsis and death in a young recipient of fresh osteochondral tissue allograft from a cadaveric donor following knee surgery that he had. The organism that was cultured from his blood was Clostridium sordellii. For those who are a little rusty on their microbiology like I was, Clostridium is an anaerobic, spore forming bacillus that is normally found in the human gastrointestinal tract.”

“the goal is to develop sterilizing methods which would eliminate bacteria, fungi and bacterial spores without adversely affecting the tissue allograft quality.”

This audible acoustic cleaning process for implants covers a method of doing exactly what Doctor Solomon said was the goal, namely a sterilizing method which would eliminate bacteria, fungi, and bacterial spores without adversely affecting the tissue allograft quality. In addition the audible acoustic cleaning process for implants eliminates viruses and removes materials from the tissue which might cause an immune reaction or inflammation.

When the body dies, the spread of bacteria through out the body is extremely rapid. Often in four to twelve hours (depends on temperature) the gut flora (gastrointestinal bacteria) have spread from the intestines throughout the tissues of the body. This, in turn, makes it very difficult to obtain donor tissues without some contamination present, not only on the tissue but inside the tissue. So a method is needed to kill and remove from the inside of tissues virtually all the various organisms that could infect a patient. It is also desirable that the cleaning and sanitization method clean the tissue of at least some of the non-essential “endogenous” materials (“endogenous” materials are those materials that are in a tissue naturally, such as: bone marrow, cells, fats, blood, etc.) in order to prevent inflammation, rejection of the tissues, or fibrous encapsulation of the allograft caused by an immune reaction or antigenicity.

It is relatively common for compound fractures resulting from severe trauma such as a motorcycle accident to require the removal and disposal of some relatively large (2 to 10 inches) pieces of bone. Because a compound fracture is where bone protrudes from the skin, the pieces of bone in the wound are typically contaminated by contact with the ground during the accident. And this bacterial contamination (an example would be the bacteria responsible for gangrene) moves into these pieces of bone at a very rapid rate, at body temperature the bacteria can totally permeate the tissue in two hours.

There was recently an “Emergency Room” episode on the Learning Channel where an orthopedic surgeon lamented about how he wished he didn't have to throw away such contaminated bone pieces, how this bone would be very useful in future surgeries if only he had a method of cleaning it. The audible sound cleaning technique could be used to treat these contaminated bone pieces in the surgical arena and place it back in the patient (either immediately or in future surgeries), greatly reducing the need for future autograft (bone typically taken from the patients hip or arm) or allograft (cadaver bone) surgeries. Because the cleaned contaminated bone sections are pieces of the original configuration, the pieces would fit together much better than any autograft or allograft, resulting in better patient outcomes, less pain for the patient, and much less cost.

Using Acoustic Energy to Clean Tissue

Acoustic energy or “sound” can be classified into three forms. It can be “audible”, where a human can hear it. This audible sound range is accepted by convention to be from 20 Hz (cycles per second) to 20,000 Hz. even though the ability to hear these frequencies varies. Then we have “infrasound”, which is generally accepted to be sound with frequencies below 20 Hz. Finally there is ultrasonic sound or “ultrasonics” where the frequency is above 20,000 Hz. The penetrating power of acoustic energy is inversely related to the frequency. We all know that the loud stereo in an apartment building only seems to really penetrate the walls in the lowest bass frequencies (20 Hz to about 300 Hz). The high “soprano” notes (about 3,000 HZ to 20,000 Hz) of the music don't come through the barrier of a solid wall, they are reflected or absorbed.

Ultrasonic sound is sound above the range of most human's hearing, above 20,000 Hz. Ultrasonic sound is easily refracted, reflected, absorbed and weakened as it goes from one medium to another, such as moving from a liquid to a solid. This weakening is especially true in materials which are not homogenous, such as bone. So any ultrasonic sound energy aimed at a solid tissue, especially bone, would lose most of its effectiveness at the surface of the tissue.

Ultrasonic sound alone can, if given extended time periods, penetrate the tissues. But extended time periods create non-homogenous treated tissues. Any antimicrobial solution does have some denaturing effect on the tissues. So, using ultrasonic sound, by the time the center of a tissue is sterile, the outside of the tissue might well be too denatured to be effective. Audible sound penetrates much better, allowing faster cycles and avoiding the denaturing effect. Another disadvantage of the ultrasonic sound is that the cavitation at the wall of the container holding the tissue, typically made of stainless steel, will rapidly pit the stainless steel and, in theory, put small amounts of the stainless steel into the tissue in microscopic form, not a desirable effect.

Another limitation of ultrasonic sound is the size of the particles being moved. While ultrasonics is generally effective on internal particles of 0.1 microns and below, audible sound is generally effective on internal particles of 50 microns and below. This all has to do with some complex rheology equations called the Navier, Stokes, Basset, Bousinesq, and Oseen equations. Without getting into these equations, suffice to say that in most of the tissue applications foreseen for this invention, cleaning particle sizes of 50 microns and below is what is desirable (for instance; blood cells are 8 microns in diameter), 0.1 microns is too small to be of much use.

Recently the internet ran a story where it talked about sinus infections and how they can be prevented in theory by the simple act of humming (“Humming may Help the Sinuses Stay Healthy”, Jennifer Warner, Web MD Medical News, Jul. 29, 2002). Acoustical energy in the audible or infrasound range makes fluids, semisolids and gases move, flow, and intermix in situ in porous solids. And this interpenetration, flow, movement and/or perfusion of debris and chemicals both in and out of the tissues is exactly what is needed to remove endogenous materials from bone and tissue and to infuse the tissue with disinfectants or antibiotics.

In summary, because audible acoustic energy induces rapid back and forth movement inside the transplant tissue, the audible acoustical energy in this tissue cleaning process does the following:

1. reduces the viscosity of the endogenous materials,

2. allows cleaning chemicals to permeate the endogenous materials,

3. promotes mixing of the chemicals and endogenous materials through turbulence,

4. removes the mixture of endogenous materials and chemicals from the tissues,

5. allows sterilizing chemicals to permeate the tissues after the endogenous materials are removed,

6. allows all the sterilizing and cleaning chemicals to be removed by pure water,

7. allows subsequent permeation by antibiotics, growth factors and the like.

Alternating Acids and Bases

The chemicals used in the preferred embodiment are sequenced initially by alternating a basic or weakly alkaline solution with a weakly acidic solution. Since fats, oils and lipids are more soluble in basic solutions and protein based compounds are more soluble in acidic solutions, this alternation of chemicals will produce faster penetrations.

Hyperbaric Oxygen

Another claim in this audible acoustic tissue cleaning process is the utilization of hyperbaric, high pressure oxygen to kill certain anaerobic bacteria. These anaerobic organisms, especially in their spore forms, are very dangerous in transplants, difficult to detect and difficult to kill by standard techniques. One anaerobic organism, Clostridium sordelli, was apparently the cause of the recent unfortunate death of a transplant recipient. The ability of pure oxygen to destroy anaerobic organisms is well documented. Specifically, organisms such as Clostridium perfringens (gangrene), C. difficile (antibiotic induced dysentery), C. tetani (tetanus), C. botulinum (botulism), C. sordellii, Prevotella buccae, etc. are destroyed by pure oxygen, even in their spore forms. The concept here is to permeate the tissue with high pressure (two to twenty atmospheres) pure oxygen (50 to 100%) in a cold (2 to 10 degrees Celsius) for 1 to 500 hours to infuse the tissue with oxygen. When the tissue is brought up to 35 to 42 degrees C. by the processing solutions, the pure oxygen dissolved in the tissue (and in any organisms in the tissue) will destroy any anaerobic bacteria or bacterial spores present.

Advantages

This audible acoustic cleaning process for implants satisfies these criteria:

1. removes or kills viruses, fungi, bacteria and other potential disease organisms, thus being capable of sterilizing tissue to the current FDA definition of “sterility”;

2. works for soft tissue and bone, whole bones or machined components;

3. does not severely impact the strength of the implants;

4. does not affect the ability of the donor tissue to act as a scaffold and encourages the growth of the host's tissue into the graft (osteoinductivity and osteoconductivity);

5, with the proper chemicals, it will not elicit an inflammatory response

6. removes endogenous materials in the transplants to prevent inflammatory and immune reactions;

7. does not require large amounts of expensive sterile water and chemicals per treatment;

8. has a treatment chamber small enough to be portable, allowing loading, unloading and chamber sterilization to take place other than in the treatment room or enclosure.

9. it is inexpensive, both in capital required and in expense monies required.

There are no expensive equipment requirements for an audible acoustic process since this process typically takes place at atmospheric pressure. There is no alternation of pressure and vacuum. Ultrasonics (acoustical energy with a frequency above 20,000 Hz) are not necessary. A vacuum is not pulled on one side of the bone while the other side of the bone is at atmospheric pressure or higher pressures. No high pressure agitation or sprays are utilized.

### SUMMARY

The basic technology to be utilized for this audible acoustic cleaning process for implants is the use of acoustical energy in the range audible to most humans (20 cycles per second to 20,000 cycles per second) to clean, sterilize, impregnate or permeate cadaver tissue to be implanted into the human body. Another use of this technology is to clean and treat contaminated bone fragments from compound fractures.

### DRAWINGS

FIG. 1 is a perspective view of the equipment to be utilized in the preferred embodiment for this process. FIG. 1 shows the shaker 14 and the treatment chamber assembly 10, 15 and 17.

FIG. 2 shows views of only the hollow treatment chamber assembly 10, 15 and 17.

### REFERENCE NUMERALS

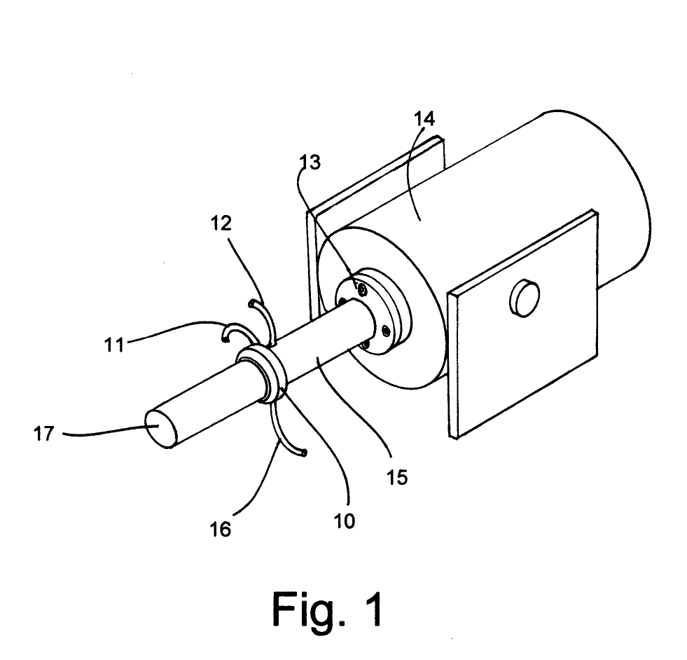

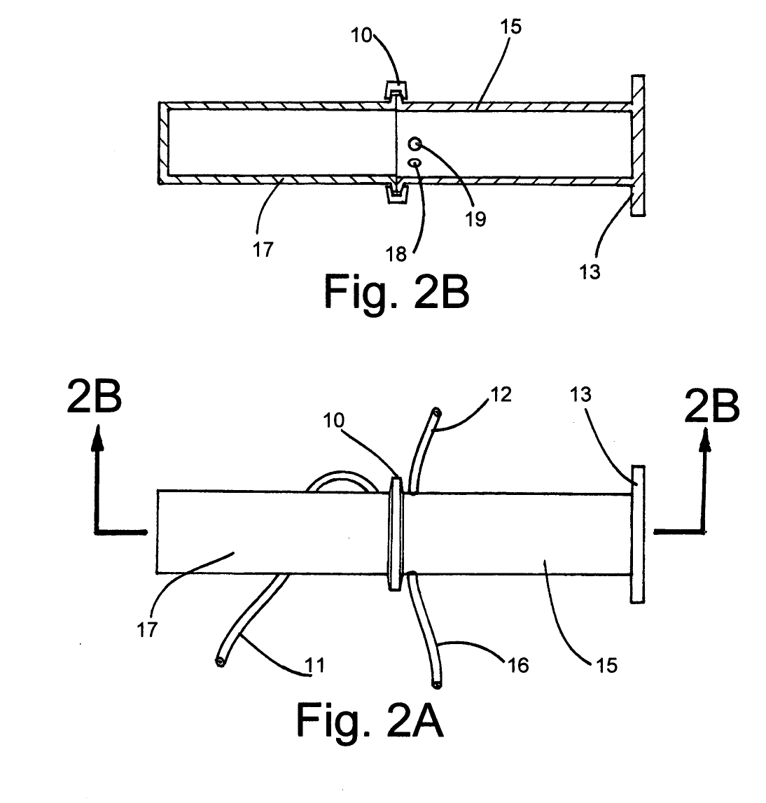

both FIG. 1 and FIG. 2 use the same numbers as follows:

10 Clamp that holds the two halves of the treatment chamber together

11 Disposable Plastic Tube for filling treatment chamber with liquids or gases

12 Disposable Plastic Tube for venting gases from the treatment chamber

13 Four bolts in a Flange that holds the treatment chamber onto the “shaker”

14 “Shaker” is a commercially available electrodynamic transducer which vibrates the assembled treatment chamber at the correct audible sound frequency

15 First Half of the Treatment Chamber, this is the first half of the hollow closed ended tubular container for treating the tissue

16 Disposable Plastic Tube that drains fluids from the treatment chamber

17 Second Half of the Treatment Chamber, is the second portion of the hollow closed ended tubular container for treating the tissue

18 Entrance Hole in the Treatment Chamber The hole through which fluids and gases enter the treatment chamber.

19 Vent Hole in the Treatment Chamber The hole through which gases in the treatment chamber escape to drain.

### DETAILED DESCRIPTION OF EQUIPMENT

Acoustical Cleaning Apparatus: The major piece of equipment in the preferred embodiment is a hollow 0.5 meter long, 12 cm inside diameter, stainless steel treatment chamber formed by clamping the two halves of the treatment chamber 15 and 17 together with the clamp 10. Clamp 10 is a standard “Tri-Clover” clamp design with standard flanges on the hollowed halves 15 and 17 of the treatment chamber. This clamp design is used in thousands of food and pharmaceutical applications and as such it is not illustrated in detail. This assembled treatment chamber 10, 15 and 17 is attached to an electrodynamic shaker 14 at one end via four bolts and a flange 13. The electrodynamic “shaker” 14 is a very powerful and sturdy source of high amplitude low frequency audible acoustic energy widely in vibrational testing (i.e. shipping tests and tests of aerospace equipment). The electrodynamic “shaker” 14 is shown not in any detail since it is widely available in commerce. The stainless steel treatment chamber assembly 10, 15 and 17 would be capable of easy removal from the “shaker” 14 to allow the tube to be sterilized in an autoclave.

There are three disposable plastic tubes 11,12, and 16 coming out from the center of the treatment chamber assembly 10, 15 and 17. Tube 11 is for filling the treatment chamber assembly 10, 15, and 17 with fluids or gas through hole 18. Tube 12 is for venting gas from the treatment chamber assembly 10, 15, and 17 from hole 19. And tube 16 is for draining fluids from the treatment chamber assembly 10, 15, and 17 (the hole is not shown, it is directly opposite hole 19).

The figures do not show all the electronics, electrical lines, controls, computers, pumps, plastic tubes, filters and apparatus associated with this process, these are all “off the shelf items. The figures only show the tooling and apparatus central to the process.

### DETAILED DESCRIPTION OF PREFERRED EMBODIMENT'S OPERATION

This is the current preferred process concept for utilizing audible acoustical energy to clean implants. Many of the steps below are optional and many alternatives will be obvious to anyone skilled in the art.

Hyperbaric Oxygen Treatment

In the preferred embodiment of the invention the tissue is treated with hyperbaric oxygen at levels of 50 to 100% and pressures of 15 to 150 psi to dissolve oxygen into the tissue prior to treatment. The tissues would be stored at 2 to 4 degrees Celsius for 8 to 80 hours prior to being put into the treatment chamber.

Tissue Loading

The three pieces 10, 15 and 17 of the half meter long stainless steel treatment chamber 10, 15 and 17 would be removed from an autoclave where they had been sterilized at 135 degrees Celsius. The partially processed or machined tissue would be placed into nylon mesh bags and then placed into one or the other of the two sides 15 and 17 of the treatment chamber. The nylon mesh bags prevent the tissues from blocking the drain 16. The first half of the treatment chamber 15 would then be assembled to the second half of the treatment chamber 17 with clamp 10. The assembled unit would then be taken to the sound proof enclosure holding the “shaker” 14.

Cleaning the Tissue with Audible Acoustic Energy

The stainless steel tube 10, 15 and 17 would be assembled to the “shaker” 14, using the end cap the flange 13 with its four bolts. The plastic line 11 to the clean air and chemicals would be attached. The gas vent line 12 would be attached, and the drain line 16 would be attached. The assembly would then be filled with a liquid and started into vibration. The electrodynamic “shaker” 14 would vibrate at about 1440 Hz (cycles per second), vibrating the treatment chamber 10, 15 and 17 along its longest axis. The liquid column inside the treatment chamber 10, 15 and 17 would then, in turn, be forced into vibration along its longest axis at about 1440 Hz. by the shaker 14. At the frequency of 1440 Hz the wavelength of the tone produced in a column of water is one meter. This treatment chamber 10, 15 and 17 is one half that length, or half a meter (inside dimensions), and the liquid column will resonate in the first harmonic, significantly increasing the sound magnitude.

Note that this frequency of 1440 Hz. will need to be adjusted to the harmonics of the treatment chamber 10, 15 and 17 as it is impossible to accurately predict the exact harmonics of a treatment chamber 10, 15 and 17, especially when the treatment chamber 10, 15 and 17 has different liquids and different tissue in it. A computer operated resonance locking scheme (not shown) and suitable measurement devices (not shown) would be used to insure this harmonic resonance is maximized during processing.

The temperature held in the tissue area would be 35 degrees Celsius to 42 degrees Celsius by using chemicals heated to this temperature. Each used solution in the processing chamber would be evacuated via the drain tube 16 via the pressure of clean air coming in through tubes 11. The air would then be evacuated out a top tube 12, the pressure for such evacuation coming from the incoming solution.

Chemical sequence A possible chemical sequence utilizing the preferred embodiment of alternating acidic solutions with basic solutions would be:

1, Add 99% ethyl alcohol, subject tissue to five minutes of audible acoustic treatment

2, Add Clean air, forcing chemicals to drain

3, Add 40% ethyl alcohol, 60% saturated basic Sodium Bicarbonate (NaHCO3) in Sterile water solution, subject tissue to five minutes of treatment.

4, Add Clean air, forcing chemicals to drain

5, Add 40% ethyl alcohol, 60% of 10% acetic acid in sterile water solution, subject tissue to five minutes of treatment.

6, Add Clean air, forcing chemicals to drain

7, Add 40% ethyl alcohol, 60% saturated basic Sodium Bicarbonate (NaHCO3) in sterile water solution, subject tissue to five minutes of treatment.

8, Add Clean air, forcing chemicals to drain

9, Add 40% ethyl alcohol, 60% of 10% acetic acid in sterile water solution, subject tissue to five minutes of treatment.

10, Add Clean air, forcing chemicals to drain

11, Add Surfactants and water (forces out clean air), subject tissue to five minutes of treatment

12, Add Clean air, forcing chemicals to drain

13, Add Surfactants and water (forces out clean air), subject tissue to five minutes of treatment.

14, Add Clean air, forcing chemicals to drain

15, Add 70% Ethyl alcohol, subject tissue to five minutes of acoustic treatment

16, Add Clean air, forcing chemicals to drain

17, Add 70% Ethyl alcohol, subject tissue to five minutes of acoustic treatment

18, Add Clean air, forcing chemicals to drain

19, Add 0.05% Sodium hypochlorite, subject tissue to five minutes of acoustic treatment

20, Add Clean air, forcing chemicals to drain

21, Add 0.1% Sodium hypochlorite, subject tissue to five minutes of acoustic treatment

22, Add Clean air, forcing chemicals to drain

23, Add 0.1% Sodium hypochlorite, subject tissue to five minutes of acoustic treatment

24, Add Clean air, forcing chemicals to drain

25, Add 0.2% Sodium Thiosulfate, subject tissue to five minutes of acoustic treatment

26, Add Clean air, forcing chemicals to drain

27, Add Pure deionized water, subject tissue to five minutes of acoustic treatment

28, Add Clean air, forcing chemicals to drain

29, Add Pure deionized water, subject tissue to five minutes of acoustic treatment

30, Add Clean air, forcing chemicals to drain

31, Add Pure deionized water, subject tissue to five minutes of acoustic treatment

32, Add Clean air, forcing chemicals to drain

33, Add Pure deionized water, subject tissue to five minutes of acoustic treatment

34, Add Clean air, forcing chemicals to drain

35, Add Pure deionized water, subject tissue to five minutes of acoustic treatment

36, Add Clean air, forcing chemicals to drain

The process delineated above would total about two hours for treating an implant. Note that the “surfactant” utilized above would consist of a water solution with 0.05 percent each of alpha olefin sulfonate, Octonxynol-9, and Sodium Laureth Sulfate. At this point all the tubes would be sealed close to the body of the treatment chamber 10, 15 and 17. The treatment chamber 10, 15 and 17 would be removed to a hood in another room and opened up, removing the cleaned and/or sterilized tissue.

### CONCLUSIONS, RAMIFICATIONS, ALTERNATE EMBODIMENTS, AND SCOPE

This use of audible sound to cleanse tissue is a highly reliable method of simply cleaning tissue or other implants. Alternatively this process can be used to clean and sterilize the implants. This audible sound acoustic cleaning technique is applicable to any transplant, graft or implant where it is desirable to clean or sanitize the inside of the implant. It is applicable to allografts (transplants from human cadavers), and xenografts (transplants from animals), and to contaminated bone fragments from compound fractures (autografts). This audible acoustic cleaning technique is also a method that can be used to perfuse such implants with antibiotics, growth factors, antioxidants (Vitamin E) or other desirable chemical entities.

The major piece of equipment, the acoustic treatment chamber used for amplifying the sound, in the preferred embodiment uses the harmonics of a simple column of a liquid. Other sound concentrating devices include parabolic sound wave concentrators; funnel shaped acoustical sound concentrators, and the use of ultrasonic transducers which convert highly directional ultrasonic sound into high intensity audible sound (so called “hypersonic sound”). Harmonics are not necessary. The equipment can consist of one chamber or many chambers converging at a common point. The equipment used for creating the high amplitude audible sound energy can be of any size from small 10 cm units to huge 3 meter units, simply depending on the application and the economies of scale desired.

The operation can proceed at any temperature from 20 degrees Celsius to 50 degrees Celsius, with 35 degrees Celsius to 40 degrees Celsius being preferred. There are many alternatives for the source of the sound, including but not limited to: all the technologies available for standard audio speakers (including underwater audio speakers), electro-pneumatic transducers, electrodynamic shakers, magneto-restrictive transducers, pneumatic devices, and piezoelectric transducers.

One interesting method of sound generation is to use the existing one half meter long treatment chamber design and replace the electrodynamic “shaker” with two piezoelectric or magnetostrictive sound producers, one at each end of the treatment chamber. If one sound producer was to emanate sound waves at 20,000 Hz and the other sound producer was to emanate sound waves at 21,440 Hz, then a “Tartini Tone” (the difference of the frequencies of two converging sound waves) of 1,440 Hz audible sound would be harmonically resonating in the chamber. This type of treatment would be especially advantageous to being used in operating rooms.

The preferred embodiment utilized hyperbaric oxygen to kill certain bacteria and their spores; this is not a necessary step in the process and can be eliminated. The hyperbaric oxygen could be anywhere from 50% to 100% oxygen and the storage temperature could range from 2 degrees Celsius to 10 degrees Celsius and the storage time range from one hour to infinity. Another known process is to perfuse the tissue with a gas such as oxygen under very high pressure (500 to 10,000 psi) and then release this pressure very rapidly. This rapid pressure release is a known and accepted method of breaking down cell walls and helping to kill organisms, the cells literally explode. This known technique could replace the hyperbaric oxygen treatment.

The cleaning and sterilizing of the tissue could all be done in one piece of equipment and in one process, as in the preferred embodiment. This audible acoustic energy process can be used for two or more separate processes on the same tissue, one being only the cleaning the tissues of endogenous materials, the other being sterilizing the tissues. And these pieces of equipment can be completely different. Indeed, one piece of equipment could utilize audible acoustic energy while the other piece of equipment could utilize an alternative process.

The preferred embodiment is done at atmospheric pressure. This process could be either done in a vacuum or at a pressure or at alternating pressures or at alternating pressure and vacuum. The preferred embodiment utilized one audible sound frequency that is constantly active. This could be replaced with multiple frequencies. The sound frequency could range from 20 Hz. to 20,000 Hz, with 300 Hz to 6,000 Hz being the optimum range. Multiple frequencies, frequency shifts, and turning on and off the audible acoustic source are all ways to increase the chaotic nature of the audible acoustic mixing and to increase the efficiency of the audible acoustic mixing.

The normal shape of a sound wave on graph paper (pressure versus time) is a sinusoidal curve, at the source of the sound. This sinusoidal shape is used at the sound source in the preferred embodiment. For this audible acoustic tissue cleaning process the shape of the audible acoustic sound pressure wave at the sound source could be sinusoidal, triangular, square, saw tooth, impulse, or any other shape. Addition of mechanical agitation and low frequency ultrasonics (20,000 to 40,000 Hz) might also improve the process.

Many different solvents and disinfectants have been used to clean tissue implants for many years. The Audible Range Acoustic Tissue Cleaning and Sanitization Process here in described can be used with any of these chemistries. Solvents can be water, ethanol, acetone, isopropyl alcohol, DMSO (dimethyl sulfoxide), ethyl acetate, esters, ethers, ketones, diethanolamine, etc. any of which can be enhanced with detergents (fatty acid esters, anionic, cationic or non-ionic surfactants, or any of the multitude of surfactants used in the food industry). Other cleaning agents can be base salts such as sodium acetate, sodium bicarbonate, potassium carbonate, and acids such as Acetic, Ascorbic, Hydrochloric, Phosphoric, and Formic. Disinfectants can be oxidizing agents (hydrogen peroxide, sodium hypochlorite, polyvinyl pyrrolidine iodine), reducing agents, guanidine hydrochloride, ionic and nonionic surfactants, detergents, acids (Acetic, Ascorbic, Hydrochloric, Phosphoric, Formic), antiviral agents, bases (Calcium Hydroxide, Sodium Hydroxide, etc.) and many others known to those skilled in the art. All these chemicals can be mixed with each other in varying proportions and used to clean and disinfect the implants in any order in any number of steps.

The preferred embodiment utilizes basic solutions being alternated with acidic solutions to clean the tissue. In actuality there are many chemistries utilized for cleaning tissue which are possible. Most of them have been used to clean tissue for many years and are apparently not patented (we could not find a tissue cleaning process patent that specified a certain chemistry).

While the above description contains many specificities, these should not be construed as limitations on the scope of the audible acoustic tissue cleaning process, but rather as an exemplification of one preferred embodiment thereof. Many other variations are possible. Accordingly the scope of the audible acoustic tissue cleaning process should be determined not by the embodiments(s) illustrated, but by the appended claims and their legal descriptions.

What is claimed:

1. A cleaning and impregnation process for tissue implants which comprises subjecting the entire tissue implant to high intensity audible sound in the range of 20 to 20,000 cycles per second in a treatment chamber filled with liquids, wherein said tissue implant is selected from a group consisting of autograft tissue, allograft tissue, xenograft tissue, tissue from a compound fracture or any combination thereof.

2. The process of claim 1 where the audible acoustic energy is augmented by ultrasonic sound in the frequency range of 20,000 to 40,000 cycles per second

3. The process of claim 1, where said treatment chamber has attached to it two higher frequency sound producing devices which, between them, produce an audible tartini tone sound, said audible tartini tone sound being the frequency obtained by subtracting one device's higher frequency from the other device's higher frequency.

4. The process of claim 1, where the liquids utilized are selected from a group consisting of ethanol, acetone, isopropyl alcohol, dimethyl sulfoxide, esters, ethers, ketones, diethanolamine, water, detergents, fatty acid esters, anionic surfactants, cationic surfactants, non-ionic surfactants, disinfectants, hydrogen peroxide, sodium hypochlorite, polyvinyl pyrrolidine iodine, reducing agents, guanidine hydrochloride, basic salts, sodium acetate, sodium bicarbonate, sodium carbonate, potassium carbonate, acids, acetic acid, ascorbic acid, hydrochloric acid, phosphoric acid, formic acid, antiviral agents, bases, calcium hydroxide, sodium hydroxide, antioxidants, vitamins, stem cells, blood plasma, serum, blood, DNA dehydrogenase and RNA dehydrogenase, enzymes, antibiotics, biologics, growth factors, proteins and any mixture of these.

5. The process of claim 1, where acidic liquids are alternated with basic liquids during the process.

6. A cleaning and impregnation process for tissue implants which comprises subjecting the entire tissue implant to harmonically intensified audible sound in the range of 20 to 20,000 cycles per second in a treatment chamber filled with liquids, wherein said tissue implant is selected from a group consisting of autograft tissue, allograft tissue, xenograft tissue, tissue from a compound fracture or any combination thereof, and where the walls of said treatment chamber are vibrated at a frequency such as to induce resonant harmonic oscillations in a liquid column in the treatment chamber at the first harmonic of the liquid column.

7. The process of claim 6, where the audible sound is between 600 and 6,000 cycles per second

8. The process of claim 6, where said treatment chamber has attached to it two higher frequency sound producing devices which, between them, produce an audible tartini tone sound, said audible tartini tone sound being the frequency obtained by subtracting one device's higher frequency from the other device's higher frequency and said tartini tone being the first harmonic of the column of liquid in said treatment chamber.

9. The process of claim 6 where the audible acoustic energy is augmented by ultrasonic sound in the frequency range of 20,000 to 40,000 cycles per second.

10. The process of claim 6, where the liquids utilized are selected from a group consisting of ethanol, acetone, isopropyl alcohol, dimethyl sulfoxide, esters, ethers, ketones, diethanolamine, water, detergents, fatty acid esters, anionic surfactants, cationic surfactants, non-ionic surfactants, disinfectants, hydrogen peroxide, sodium hypochlorite, polyvinyl pyrrolidine iodine, reducing agents, guanidine hydrochloride, basic salts, sodium acetate, sodium bicarbonate, sodium carbonate, potassium carbonate, acids, acetic acid, ascorbic acid, hydrochloric acid, phosphoric acid, formic acid, antiviral agents, bases, calcium hydroxide, sodium hydroxide, antioxidants, vitamins, stem cells, blood plasma, serum, blood, DNA dehydrogenase and RNA dehydrogenase, enzymes, antibiotics, biologics, growth factors, proteins and any mixture of these.

11. A method of partially sterilizing tissues for implanting where the tissue is treated in 50 to 100% pure oxygen at pressures of 2 to 20 atmospheres and temperatures of 2 to 10 degrees Celsius for more than one hour.
